(19) United States
(12) Patent Application Publication
(10) Pub. No.: US 2024/0006041 A1
(43) Pub. Date: Jan. 4, 2024

(54) SYSTEMS AND METHODS FOR MOBILE INVESTIGATIONAL PRODUCT MANAGEMENT

(71) Applicant: IQVIA Inc., Durham, NC (US)

(72) Inventors: Jonathan Walter, Durham, NC (US); Sumanta Sharma, Durham, NC (US); Bradley Kwon, Durham, NC (US); Brian Wilson Williams, Apex, NC (US)

(21) Appl. No.: 18/217,537

(22) Filed: Jul. 1, 2023

Related U.S. Application Data
(60) Provisional application No. 63/357,741, filed on Jul. 1, 2022.

(51) Int. Cl. G16H 20/10; G16H 50/20; G16H 10/20

(52) U.S. Cl. CPC G16H 20/10; G16H 50/20; G16H 10/20

(57) ABSTRACT

A method includes receiving data images of patient medications. The method also creates a training set using the received data images. The method also includes training a machine learning system using the training set. The machine learning system is trained to monitor shipment and inventory of the patient medications, patient enrollment in medical trials, and a distribution of the patient medications. The method also includes applying the trained machine learning system with monitoring results of the shipment and inventory of the patient medications, the patient enrollment in the medical trials, and the distribution of the patient medications.

### CROSS-REFERENCE TO RELATED APPLICATION

This applications claims priority to U.S. Provisional Application Ser. No. 63/357,741, filed Jul. 1, 2022, entitled “Mobile IP,” the disclosure of which is hereby expressly incorporated herein by reference in its entirety.

### TECHNICAL FIELD

The present disclosure generally relates to reducing manual review of shipment and inventory of patient medications, patient enrollment in medical trials, and distribution of the patient medications.

### BACKGROUND

Current systems that monitor patient medications and patient enrollment in medical trials often require clinical research associates to monitor the distribution of patient medications. The clinical research associates may also have to monitor the accuracy of the patient medication distribution. Further, the clinical research associates may also have to manually check the accuracy of the shipment of any patient medications and the enrollment of patients in medical trials.

The accuracy of the shipment and inventory may not be ensured based on a manual review by clinical research associates. Errors are more likely to occur by manual review. Further, mistakes can occur with the distribution of patient medications, and with which patients are enrolled in which medical trails. Since the clinical research associates may perform a manual review in all phases, mistakes are more likely to occur.

Another issue with the primary use of clinical research associates is the greater time it can take for manual review of each phase of the process. It may take longer for clinical research associates to review the inventory of medications, and longer to verify that the right patients were enrolled in the proper medical trials. It may also take longer to verify patient medications being given in the medical trials.

Another issue is how much burden is placed on clinical research associates to monitor each phase of the process. The clinical research associates may often be unduly burdened with having to provide manual review for each phase of the process.

Accordingly, a more efficient system is needed to reduce the burden on clinical research associates. Moreover, a system is needed that takes less time to ensure accuracy at each phase of the process. Further, a system is also needed that significantly reduces the level of mistakes that occur with current monitoring systems.

### SUMMARY

The following summary is provided to facilitate an understanding of some of the features of the disclosed embodiments and is not intended to be a limiting description. A full appreciation of the various aspects of the embodiments disclosed herein can be gained by taking the specification, claims, drawings, and abstract as a whole. The aforementioned aspects and other objectives are now achieved as described herein.

In an embodiment, a computer-implemented method comprises receiving data images of patient medications from one or more databases. The method also includes creating a training set using the received data images of patient medications. Further, the method includes creating a training set using the received data images of patient medications. The machine learning system is trained to monitor shipment and inventory of the patient medications, patient enrollment in one or more medical trials, and a distribution of the patient medications in the one or more medical trials. The method also includes applying the trained machine learning system with monitoring results of the shipment and inventory of the patient medications. The applying of the trained machine learning system also includes using the monitoring results of patient enrollment in the one or more medical trials. The applying of the trained machine learning system also includes using the monitoring results of the distribution of the patient medications in the one or more medical trials.

The method also includes determining an accuracy level of the monitoring results of the shipment and inventory of the patient medications.

The method also includes determining whether to re-train the machine learning system based on an accuracy level of the monitoring results in relation to the patient enrollment in the one or more medical trials.

In an embodiment, a computer program product is configured to receive data images of patient medications from one or more databases. The computer program product also creates a training set using the received data images of patient medications. Further, the computer program product trains a machine learning system using the training set. The machine learning system is trained to monitor shipment and inventory of the patient medications. The machine learning system is also trained to monitor patient enrollment in one or more medical trials. Further, the machine learning system is trained to monitor a distribution of the patient medications in the one or more medical trials. The computer program product also applies the trained machine learning system with monitoring results of the shipment and inventory of the patient medications, patient enrollment in the one or more medical trials, and the distribution of the patient medications in the one or more medical trials.

The machine learning system is re-trained based on an accuracy level of the monitoring results of the distribution of patient medications.

The monitoring results of the patient enrollment in the one or more medical trials satisfy an accuracy threshold.

In an embodiment, a system includes a memory to store instructions. The system also includes one or more processors to execute the instructions. The one or more processors perform operations to receive data images of patient medications from one or more database. The operations also create a training set using the received data images of patient medications. Further, the operations also train a machine learning system using the training set. The machine learning system is trained to monitor shipment and inventory of patient medications, patient enrollment in one or more medical trials, and a distribution of the patient medications in the one or more medical trials. Further, the operations include applying the trained machine learning system with monitoring results of the shipment and inventory of the patient medications. In addition, the operations also include applying the trained machine learing system with the monitoring results of the patient enrollment in the one or more medical trials. Further, the operations include applying the trained machine learning system with monitoring results of the distribution of the patient medications in the one or more medical trials.

A determination is made as to whether the machine learning system needs to be re-trained based on the monitoring results of the distribution of the patient medications in the one or more medical trials.

A level of source data verification for clinical research associates is reduced.

### BRIEF DESCRIPTION OF THE FIGURES

The accompanying figures, in which like reference numerals refer to identical or functionally similar elements throughout the separate views and which are incorporated in and form a part of the specification, further illustrate the present disclosure and, together with the detailed description, serve to explain the principles of the present disclosure.

FIG. 1 illustrates a computing system in accordance with an embodiment of the present disclosure;

FIG. 2 illustrates a source and monitoring system in accordance with an embodiment of the present disclosure;

FIG. 3 illustrates a flow diagram of target benefit streams in accordance with an embodiment of the present disclosure; and

FIG. 4 illustrates a flow chart in accordance with an embodiment of the present disclosure;

Unless otherwise indicated illustrations in the figures are not necessarily drawn to scale.

### DETAILED DESCRIPTION

Subject matter will now be described more fully herein after with reference to the accompanying drawings, which form a part hereof, and which show, by way of illustration, specific example embodiments. Subject matter may, however, be embodied in a variety of different forms and, therefore, covered or claimed subject matter is intended to be construed as not being limited to any example embodiments set forth herein, example embodiments are provided merely to be illustrative. Likewise, a reasonably broad scope for claimed or covered subject matter is intended. Among other issues, subject matter may be embodied as methods, devices, components, or systems. The followed detailed description is, therefore, not intended to be interpreted in a limiting sense.

Throughout the specification and claims, terms may have nuanced meanings suggested or implied in context beyond an explicitly stated meaning. Likewise, phrases such as “in one embodiment” or “in an example embodiment” and variations thereof as utilized herein may not necessarily refer to the same embodiment and the phrase “in another embodiment” or “in another example embodiment” and variations thereof as utilized herein may or may not necessarily refer to a different embodiment. It is intended, for example, that claimed subject matter include combinations of example embodiments in whole or in part.

In general, terminology may be understood, at least in part, from usage in context. For example, terms such as “and,” “or,” or “and/or” as used herein may include a variety of meanings that may depend, at least in part, upon the context in which such terms are used. Generally, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures, or characteristics in a plural sense. Similarly, terms such as a “a,” “an,” or “the”, again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

One having ordinary skill in the relevant art will readily recognize the subject matter disclosed herein can be practiced without one or more of the specific details or with other methods. In other instances, well-known structures or operations are not shown in detail to avoid obscuring certain aspects. This disclosure is not limited by the illustrated ordering of acts or events, as some acts may occur in different orders and/or concurrently with other acts or events. Furthermore, not all illustrated acts or events are required to implement a methodology in accordance with the embodiments disclosed herein.

Unless defined otherwise, all technical and scientific terms used herein have the same meanings as commonly understood by one of ordinary skill in the art to which the disclosed embodiments belong. Preferred methods, techniques, devices, and materials are described, although any methods, techniques, devices, or materials similar or equivalent to those described herein may be used in the practice or testing of systems and methods of the present disclosure.

Although claims have been included in this application to specific enumerated combinations of features, it should be understood the scope of the present disclosure also includes any novel feature or any novel combination of features disclosed herein.

References “an embodiment,” “example embodiment,” “various embodiments,” “some embodiments,” etc., may indicate that the embodiment(s) so described may include a particular feature, structure, or characteristic, but not every possible embodiment necessarily includes that particular feature, structure, or characteristic.

Headings provided are for convenience and are not to be taken as limiting the present disclosure in any way.

Each term utilized herein is to be given its broadest interpretation given the context in which that term is utilized.

The following paragraphs provide context for terms found in the present disclosure (including the claims):

The transitional term “comprising”, which is synonymous with “including,” “containing,” or “characterized by,” is inclusive or open-ended and does not exclude additional, unrecited elements or method steps. See, e.g., Mars Inc. v. H.J. Heinz Co., 377 F.3d 1369, 1376, 71 USPQ2d 1837, 1843 (Fed. Cir. 2004) (“[L]ike the term ‘comprising,’ the terms ‘containing’ and ‘mixture’ are open-ended.”). “Configured to” or “operable for” is used to connote structure by indicating that the mechanisms/units/components include structure that performs the task or tasks during operation. “Configured to” may include adapting a manufacturing process to fabricate components that are adapted to implement or perform one or more tasks.

“Based On.” As used herein, this term is used to describe factors that affect a determination without otherwise precluding other or additional factors that may affect that determination. More particularly, such a determination may be solely “based on” those factors or based, at least in part, on those factors.

All terms of example language (e.g., including, without limitation, “such as”, “like”, “for example”, “for instance”, “similar to”, etc.) are not exclusive of other examples and therefore mean “by way of example, and not limitation . . . ”.

A description of an embodiment having components in communication with each other does not infer that all enumerated components are needed.

A commercial implementation in accordance with the scope and spirit of the present disclosure may be configured according to the needs of the particular application, whereby any function of the teachings related to any described embodiment of the present disclosure may be suitably changed by those skilled in the art.

The flowcharts and diagrams in the figures illustrate the architecture, functionality, and operation of possible implementations of systems and methods according to various embodiments. Functions noted in the block may occur out of the order noted in the figures. For example, two blocks shown in succession may, in fact, be executed substantially concurrently, or the blocks may sometimes be executed in the reverse order, depending upon the functionality involved.

Further, any sequence of steps that may be described does not necessarily indicate a condition that the steps be performed in that order. Some steps may be performed simultaneously.

Embodiments of the present disclosure provide digital source and monitoring delivery. The Mobile IP provides automation of the monitoring of shipment and inventory of patient medications. In addition, the Mobile IP automates the monitoring of the patient enrollment in medical trials and the dispensation of the patient medications in the medical trials.

Input data in the form of data images of patient medications is received by the system. A computer processing unit (CPU) within the system creates a training data set from the received input data. The training data set is used to train a machine learning system to monitor results for the shipment and inventory of the patient medications, the patient enrollment in the medical trials, and the dispensation of the patient medications.

The monitoring results may be verified for accuracy. Moreover, the accuracy may be determined by a comparison of an expected result versus an actual result. At each phase, the monitoring results may be compared to an accuracy threshold with the actual monitoring results being compared to the expected or predicted monitoring results. The monitoring results may be compared to the accuracy threshold to ensure accuracy of the monitoring results at each phase. If the monitoring results do not satisfy the accuracy threshold at any phase, the Mobile IP then re-trains the machine learning system until the accuracy threshold is satisfied. Moreover, the machine learning system can be re-trained for the required amount of iterations until accuracy is satisfied at each phase, wherein the actual monitor results satisfy the expected or predicted monitoring results.

The monitoring results at each phase are initiated into the trained machine learning system to enable the machine learning system to produce output. The machine learning system produces the output data that shows automation of the shipment and inventory of the patient medications, automation of the patient enrollment in the medical trials, and automation of the dispensation of the patient medications in the medical trials. The monitoring level of clinical research associates (CRA's) is eliminated or substantially reduced due to the automation.

The automation of each phase provides many benefits to the system, such as improvements in productivity and quality within the Mobile IP, improvements to the Trial Master File (TMF) compliance and new business, and improvements with sites working with the company as well.

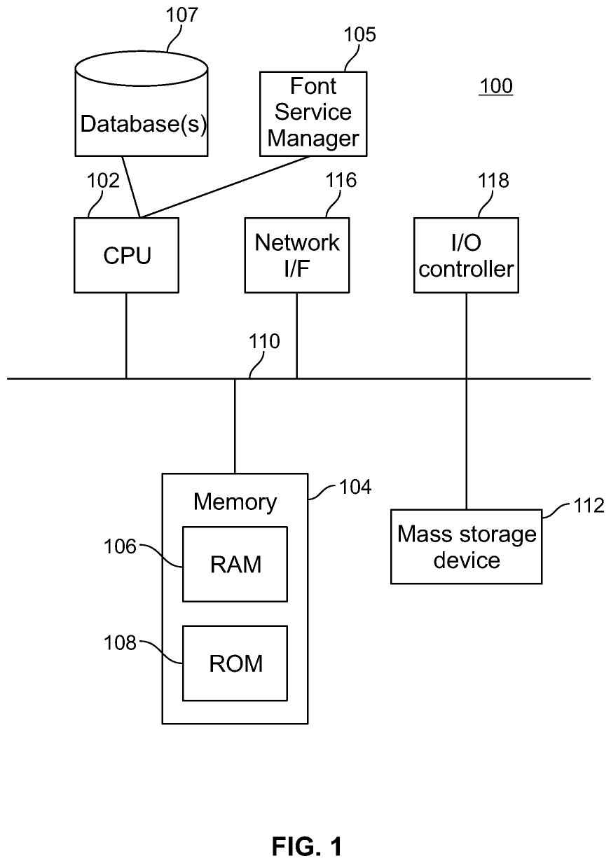

FIG. 1 illustrates a computing system 100. The computing system 100 includes a computer-processing unit (CPU) 102. The CPU 102 is connected to memory 104 with a random access memory (RAM) 106 and read only memory (ROM) 108. The CPU 102 is also connected to one or more databases 107 and a font service manager 105. A communication interface 110 enables the CPU 102 to be connected to a network 116 and a controller 118. A mass storage device 112 is also coupled to a controller 118 by way of the network 110.

In FIG. 1, in an embodiment, the CPU 102 can collect one or more data images of patient medications. The data images can be collected from a plurality of databases. The patient medications can be those used for, or in connection with, the treatment of a broad spectrum of ailments, diseases, disorders, and the like. For example, the patient medications can be used for the treatment of simple diseases, disorders, and ailments. In addition, the patient medications can be used for the treatment of other moderate ailments, disorders, and diseases. Moreover, the patient medications can also be used for the treatment of more serious diseases and terminal ailments that affect patients in any region. Accordingly, the data images of patient medications may include images of medications that are used for, or in connection with, the treatment of a broad spectrum of various ailments, disorders, and diseases for patients in any region.

Referring to FIG. 1, the CPU 102 can create a training set that includes the received data images of the patient medications that treat a broad spectrum of ailments, disorders, and diseases for patients in any region. The training set can be used to train a machine learning system within the computing system 100. The CPU 102 thereby trains the machine learning system within the computing system 100 with the training set. The machine learning system is trained to monitor results of shipment and inventory of patient medications. The patient medications are those used to treat patients during a series of medical trials. In addition, the machine learning system may also be trained to monitor the enrollment of patients in the medical trials. Further, the machine learning system may also be trained to monitor the distribution of the patient medications during the medical trials.

Referring to FIG. 1, once the machine learning system has been trained with the training data set, the trained machine learning system can be applied. At each phase, the monitoring results are compared to expected or predicted monitoring results to determine if the monitoring results satisfy an accuracy threshold. As such, the CPU 102 can compare the monitoring results from the shipment and inventory of the patient medications, and the patient enrollment in the medical trails to the accuracy threshold. Moreover, the CPU 102 can determine if the monitoring results are at the required accuracy level. If the monitoring results are not accurate and do not meet the expected monitoring results, then the CPU 102 can determine that the machine learning system needs to be re-trained to produce more accurate monitoring results. In addition, the CPU 102 can also compare the monitoring results from the distribution of the patient medications in the medical trials to the expected monitoring results. The CPU 102 will compare the monitoring results from the distribution of the patient medications to the expected monitoring results to determine if the accuracy threshold is satisfied.

In FIG. 1, if the CPU 102 determines that the monitoring results from the dispensation of the medications do not satisfy the expected monitoring results and the accuracy threshold, then the CPU 102 can determine that the machine learning system should be re-trained. The CPU 102, with the dispensation phase, and with the earlier phases, can re-train the machine learning system to produce more accurate monitoring results to satisfy the expected monitoring results and the accuracy threshold. As such, the CPU 102 can re-train the machine learning system to provide more accurate results of the shipment and inventory of the patient medications, the patient enrollment in the medical trials, and the distribution of the patient medications in the medical trials. Moreover, the CPU 102 can re-train the machine learning system in all phases of the process until the monitoring results at each phase satisfy the accuracy threshold.

In FIG. 1, when the monitoring results have satisfied the accuracy threshold at each phase, the CPU 102 uses the monitoring results from each phase, and imitates the monitoring results onto the trained machine learning system. The trained machine learning system that receives the input of the monitoring results can produce output that shows complete automation. The shipment inventory of the patient medications can be fully automated by the systems and methods of the present disclosure. Further, the enrollment of patients in medical trials and distribution of the medications in the medical trials can also be fully automated by the systems and methods of the present disclosure. As such, with the automation at each phase, the manual review at each phase by CRA's is eliminated and/or substantially reduced. Accordingly, the use of CRA's can be eliminated or substantially reduced at each phase of the process.

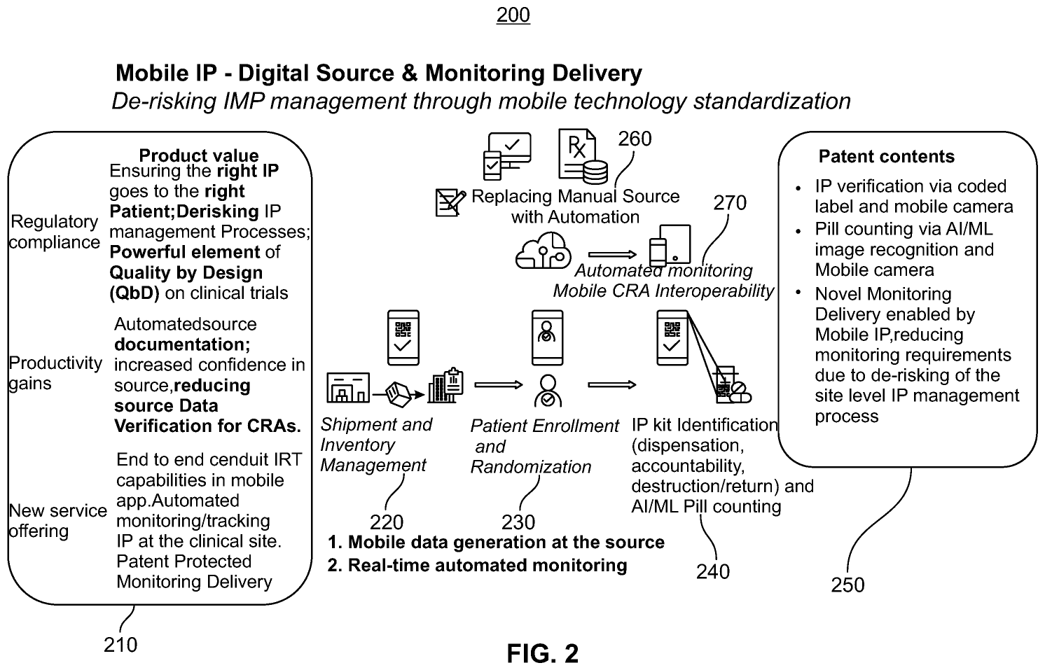

Referring to FIG. 2, a system 200 is illustrated in which a manual source automation replaces the review of clinical research associates. The system 200 monitors the results of the shipment and inventory of patient medications, the patient enrollment in medical trials, and the distribution of the patient medications in the medical trials. The monitoring results at each phase are compared to expected monitoring results to determine if the accuracy threshold for the monitoring results has been satisfied. The system 200 re-trains the machine learning system that is configured within the system 200 when the monitoring results are inaccurate and do not satisfy the expected monitoring results and the accuracy threshold. Moreover, the system 200 can re-train the machine learning system if the monitoring results are inaccurate at any phase of the process. The system 200 can also apply the monitoring results to the trained machine learning system to produce output that indicates an automated monitoring of each phase with the required accuracy at each phase.

In FIG. 2, the system 200 includes a product value 210. The product value 210 can include regulatory compliance. The risk in IP management processes is also be reduced. Quality by design in clinical trials also occurs. The product value 210 can also include productivity gains. The productivity gains can include automated source documentation. There will also be confidence in the automated process and a reduced level of source data verification required by CRA's. The product value 210 will also include a new service offering. The new service offering can include end to end interactive response technology (IRT) capabilities. Moreover, there can be automated monitoring and tracking of IP at the clinical site. The patients are protected and the delivery of any patient medications is monitored. The product value 210 can also represent the benefits that can be provided, by the system 200.

Referring to FIG. 2, the system 200 can receive data images of patient medications that are received from one or more databases. Moreover, the computing system described in FIG. 1 can receive the data images of patient medications from the one or more databases. The received data images can be used as training data. The CPU creates a training data set of the received data images of patient medications. The training data set is used to train a machine learning system configured within the system 200. Accordingly, the training data set is then inputted into the machine learning system to train the machine learning system to monitor results. Moreover, the machine learning system is trained to monitor results of the shipping and inventory of patient medications. The machine learning system is also trained to monitor the enrollment of patients in medical trials. Further, the machine learning system is trained to monitor the distribution of the patient medications in the medical trials.

Referring again to FIG. 2, the machine learning system monitors the results of the shipment and inventory 220 of the patient medications. The monitoring results are compared to the expected or predicted results to determine if the accuracy threshold is satisfied. An assessment is made as to whether the shipment and inventory 220 of the patient medications are going to the right locations. A determination is made as to how many of the shipments are going to the right patients and/or health related facilities. Moreover, the system 200 determines if it is accurately monitoring where each patient medication of the shipment and inventory 220 is being sent. The system 200 thereby determines if the accuracy of the monitoring results satisfy the accuracy threshold. Similarly, the system 200 also determines if the machine learning system should be re-trained if the monitoring results are less than accurate and do not satisfy the accuracy threshold.

In FIG. 2, the system 200 also monitors the patients enrolling or patient enrollment and randomization (patient enrollment) 230 in the medical trials. The system identifies whether the patients are enrolling in the correct medical trials. The correct medical trials are the medical trials that are designed to treat and/or address the ailments of the appropriate patients. The system 200 thereby identifies whether the patients have enrolled in the appropriate medical trials for treatment. The system 200 can also identify if there are an appropriate number of medical trials for the patients. Further, the system 200 also identifies whether it is accurately monitoring the patient enrollment 230 in the medical trials as they are occurring in real-time. The system 200 determines whether the monitoring results of the patient enrollment 230 in the medical trials satisfy the expected monitoring's results and the accuracy threshold. If the monitoring results do not satisfy the accuracy threshold, then the system 200 determines whether the machine learning system needs to be re-trained to produce more accurate monitoring results with the patient enrollment 230.

Still referring to FIG. 2, the system 200 also determines the accuracy of the monitoring results of the dispensation 240 of the patient medications in the medical trials. The system 200 identifies if it is accurately monitoring the dispensation of the patient medications to the patients in the medical trials. Further, the system 200 also identifies if the patients are receiving the proper or correct patient medications. As such, the system 200 will compare the monitoring results of the dispensation 240 to the expected monitoring results and accuracy threshold to determine if the accuracy threshold has been satisfied. If the accuracy threshold has been satisfied, then the machine learning system may not require further training to monitor the dispensation 240. In contrast, if the accuracy threshold is not satisfied, the system. 200 may determine that the machine learning system should be retrained to more accurately monitor the dispensation 240. At each phase, the system 200 can perform the necessary iterations and re-iterations to re-train the machine learning system to ensure that the monitoring results at each phase satisfy the expected/predicted monitoring results and the accuracy threshold.

In FIG. 2, the monitoring results from the shipment and inventory 220, patient enrollment 230, and dispensation 240 are applied to the train machine learning system to produce the output. The output of the machine learning system indicates a fully automated system 200. With the fully automated system 200, the manual source review at each stage by the CRA's has been removed or significantly reduced. The accuracy of the fully automated system. 200 satisfies the required accuracy threshold to eliminate the need for manual review by CRA's. As such, the fully automated system 200 can satisfy the accuracy threshold at each phase from the shipment and inventory 220, patient enrollment 230, to the dispensation 240.

Referring to FIG. 2, the patent contents 250 are also shown. The patent contents include IP verification via coded labels on the patient medications. The patent contents 250 also include a mobile camera in relation to the monitoring of the shipment and inventory 220, patient enrollment 230, and dispensation 240. The patent contents 250 also includes pill counting in relation to the patient medications. The machine learning system uses image recognition and a mobile camera to identify the pill count of the patient medications at each phase. The machine learning system enables novel automated monitoring and significantly reduces the monitoring requirements of the CRA's. As such, the CRA's have a reduced level of responsibility with respect to monitoring each phase and do not have to perform significant levels of manual review and monitoring. The dependence on the CRA's is reduced by reducing (de-risking) the risk of the site level IP management process.

In FIG. 2, the system 200 is configured to replace manual source review by CRA's with automation 260. The automation 260 replaces the manual review of the CRA's when the monitoring results of the shipment and inventory 220, patient enrollment 230, and dispensation 240 each have a high level of accuracy that satisfy the expected or predicted monitoring results and the accuracy threshold. The automation 260 occurs when the monitoring becomes fully accurate at all phases. When the monitoring results are accurate at every phase and satisfy the accuracy threshold, the manual source review of the CRA's is dramatically reduced and/or eliminated.

Referring to FIG. 2, automated monitoring 270 is also shown. With automated monitoring 270, the Mobile IP can make the CRA manual source review unnecessary. When the monitoring results at each phase of the shipment and inventory 220, patient enrollment 230, and dispensation 240 satisfy the accuracy threshold, the process can be fully automated. As such, the automated monitoring 270 eliminates and/or drastically reduces the need for CRA's to perform review at either the shipment and inventory 220, patient enrollment 230, and/or dispensation 240. With the reduced need of the CRA's, mistakes and loss in time and productivity are reduced or eliminated. The automated monitoring 270 ensures that the process will have completely automated monitoring at all phases.

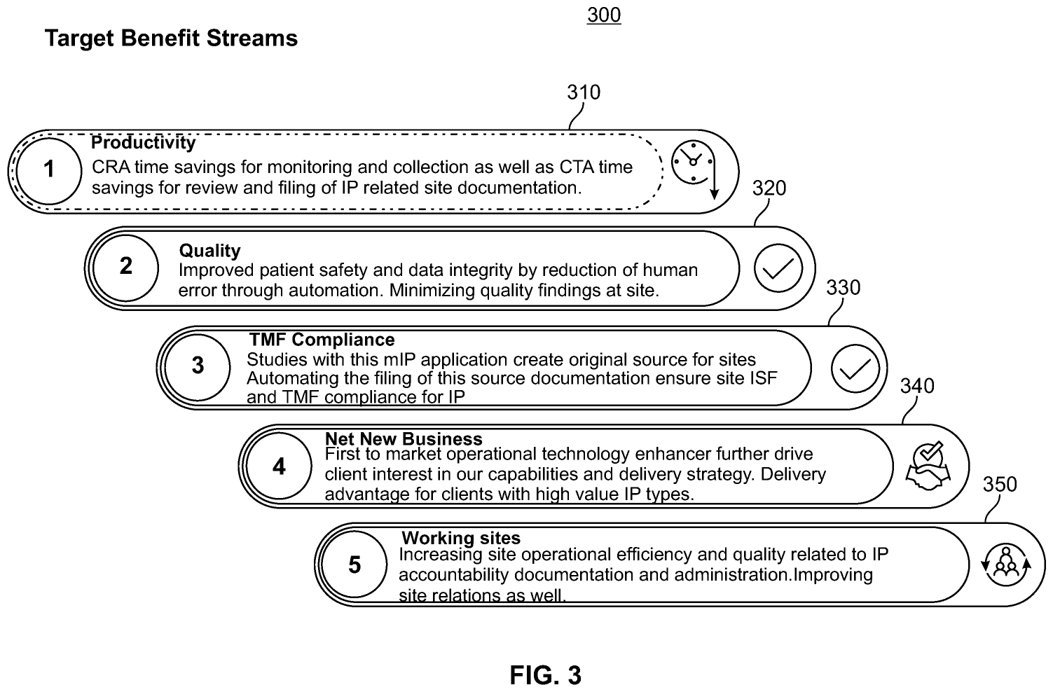

In FIG. 3, a flow diagram of target benefit streams 300 is illustrated based on the automated machine learning system. Productivity 310 includes benefits due to the automation. Productivity 310 includes time savings for CRA's. The CRA's spend much less time monitoring and collecting monitoring data of the various phases of the process. The CRA's also have time savings for their review and filing of IP related site documentation. The target benefit streams 300 also include quality 320. Quality 320 includes improved patient safety and data integrity. Moreover, the improved patient safety and data integrity occur by reducing the need for the CRA's to perform review through the automation. The improved patient safety can be the patients receiving the proper medications and being enrolled in the appropriate medical trials. The improved patient safety and data integrity also occur by minimizing the quality findings at the patient site through automation.

Referring to FIG. 3, the target benefit streams also include Trial Master File (TMF) compliance 330. IMF compliance 330 includes studies with the Mobile IP application that create an original source for patient sites. Further, TMF compliance 330 includes automated filing of source documentation. The automated filing of the source documentation will ensure site compliance for the IP. The target benefit streams will further include Net New Business 340. Net New Business 340 includes first to market operational technology enhancer. The technology enhancer increases client interest in the capabilities of the automated process and delivery strategy of the automated process. Net New Business 340 also includes a delivery advantage for clients with high value IP.

In FIG. 3, sites 350 working with the automated process have increased operational efficiency. The quality of the sites 350 is improved. The sites 350 have increased operational efficiency due to the automation at each phase from the shipment and inventory, patient enrollment, and the dispensation of the patient medications. The quality of the IP accountability, documentation, and administration is also be greatly improved due to the automation of the machine learning system. The reduced reliance on the CRA's eliminates unduly burdening the CRA's with tasks, and also eliminates mistakes that occur from manual review. The relations of the sites 350 improved as well. Overall, the fully automated process improves the company sites 350 in virtually all phases of functionality and operability.

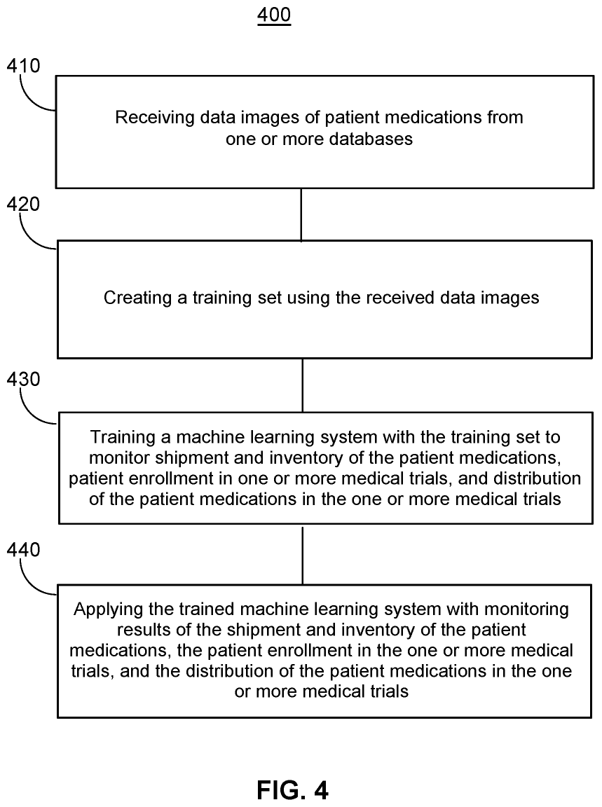

FIG. 4 is a flowchart 400 illustrating overall processing steps carried out by the system of the present disclosure in accordance with embodiments thereof. At step 410, the computing system receives data images of patient medications from one or more databases. As described herein, the patient medications can range from medications for minor ailments, to moderate ailments, to terminal diseases and other more serious ailments. Accordingly, the computing system receives data images of the spectrum of patient medications from one or more databases.

In FIG. 4, at step 420, the computing system creates a training set using the received data images. The training set includes the images of the patient medications that treat minor ailments, moderate ailments, and terminal ailments and other serious diseases.

Referring to FIG. 4, at step 430, the training set is used to train a machine learning system within the computing system. The machine learning system is trained to monitor shipment and inventory of patient medications. Further, the machine learning system is also trained to monitor patient enrollment in one or more medical trials. In addition, the machine learning system is also trained to monitor a distribution of the patient medications in the one or more medical trials.

In FIG. 4, at step 440, the trained machine learning system is applied in connection with monitoring results from the shipment and inventory of the patient medications. In addition, the trained maching learning system is also applied in connection with monitoring results with the patient enrollement in the one or more medical trials. Further, the trained machine learning system is also applied in connection with monitoring results from the distribution of the patient medications in the one or more medical trials.

Those skilled in the art will appreciate that the example embodiments are non-exhaustive and that embodiments other than that described here may be included without departing from the scope and spirit of the presently disclosed embodiments.

The process of creating a fully automated system for shipment and inventory of patient medication, patient enrollment in medical trials, and dispensation of the patient medications can occur in the embodiments cited above. A system can receive input of data images of patient medications. The system can also create a training set from the received input data. The training set can be used to train the machine learning system. The machine learning system can be trained to provide monitoring results of the shipment and inventory of the patient medications, the patient enrollment in the medical trials, and the dispensation of the patient medications in the medical trials.

The monitoring results of each phase are compared for accuracy. In various embodiments, the accuracy for each phase can be compared to the expected monitoring results, and thereby the accuracy threshold. If any of the monitoring results do not satisfy the accuracy threshold, the system can initiate a re-training of the machine learning system until the accuracy threshold is satisfied for each phase of the process. Accordingly, the system can re-train the machine learning system until the accuracy threshold is satisfied at each phase of the process.

The monitoring results for each phase are then initiated into the trained machine learning system. Once the machine learning system has been trained and re-trained to the necessary iterations to enable the monitoring results to satisfy the accuracy threshold at each phase, the monitoring results are initiated into the trained machine learning system. The trained machine learning system will then produce output results that facilitate a fully automated system at each phase. The shipment and inventory of the patient medications can be fully automated. The need for monitoring for accuracy by CRA's is eliminated or substantially reduced. Similarly, the patient enrollment in the medical trials and the distribution of the patient medications in the medical trials can be automated, with a substantially reduced or eliminated need for monitoring by CRA's.

The automation of each phase of the process provides target benefit streams. In particular, productivity is improved. There will be time savings for the CRA's for monitoring, collecting, reviewing, and filing IP related site documentation. There is less time required for review due to the automation. The quality is also be improved. The automation reduces mistakes due to human error that leads to improved patient safety and data integrity. The TMF compliance is also benefitted from the automation for each phase. In particular, original sources for sites will be created. The automated filing of the source documentation will ensure the IMF compliance.

Other benefitted streams include new net business. First to market operational technology increases client capabilities due to the capabilities and delivery strategy of the automated process. There is also a delivery advantage from the automated process as well. Another benefitted stream includes sites working with the company. Moreover, there is increased site efficiency. The quality related to the IP accountability, documentation, and administration is improved. Site relations are also be improved.

Overall, reducing the need for monitoring by CRA's and creating an automated process to monitor all phases from the shipment and inventory, to patient enrollment, to the distribution of the patient medications provides many continuous benefits. The benefits improve the overall operability and functionality of the system.

All references, including granted patents and patent application publications, referred herein are incorporated herein by reference in their entirety.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Various aspects of the present disclosure have been described above by way of illustration, and the specific embodiments disclosed are not intended to limit the present disclosure to the particular forms disclosed. The particular implementation of the system provided thereof may vary depending upon the particular context or application. The present disclosure is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

The corresponding structures, materials, acts, and equivalents of all means or step plus function elements in the claims below are intended to include any structure, material, or act for performing the function in combination with other claimed elements as specifically claimed.

What is claimed is:

1. A computer-implemented method comprising:
receiving data images of patient medications from one or more databases;
creating a training set using the received data images of patient medications;
training a machine learning system using the training set, wherein the machine learning system is trained to monitor shipment and inventory of the patient medications, patient enrollment in one or more medical trials, and a distribution of the patient medications in the one or more medical trials; and
applying the trained machine learning system with monitoring results of the shipment and inventory of the patient medications, the patient enrollment in the one or more medical trials, and the distribution of the patient medications in the one or more medical trials.

2. The method of claim 1, further comprising:
determining an accuracy level of the monitoring results of the shipment and inventory of the patient medications.

3. The method of claim 1, further comprising:
determining whether to re-train the machine learning system based on an accuracy level of the monitoring results in relation to the patient enrollment in the one or more medical trials.

4. The method of claim 1, further comprising:
re-training the machine-learning system based on the monitoring results of the shipment and inventory.

5. The method of claim 1, further comprising:
identifying whether one or more patients received correct patient medications.

6. The method of claim 1, further comprising:
monitoring the enrollment of patients in the one or more medical trials in multiple time intervals.

7. The method of claim 1, further comprising:
replacing a manual review of the monitoring results of the patient enrollment in the one or more medical trials.

8. A computer program product comprising a tangible storage medium encoded with processor-readable instructions that, when executed by one or more processors, enable the computer program product to:
receive data images of patient medications from one or more databases;
create a training set using the received data images of patient medications;
train a machine learning system using the training set, wherein the machine learning system is trained to monitor shipment and inventory of the patient medications, patient enrollment in one or more medical trials, and a distribution of the patient medications in the one or more medical trials; and
apply the trained machine learning system with monitoring results of the shipment and inventory of the patient medications, patient enrollment in the one or more medical trials, and the distribution of the patient medications in the one or more medical trials.

9. The computer program product of claim 8, wherein the machine learning system is re-trained based on an accuracy level of the monitoring results of the distribution of patient medications.

10. The computer program product of claim 8, wherein the monitoring results of the patient enrollment in the one or more medical trials satisfy an accuracy threshold.

11. The computer program product of claim 8, wherein a determination is made as to whether to re-train the machine learning system based on the monitoring results of the shipment and inventory of the patient medications.

12. The computer program product of claim 8, wherein inaccurate data is identified within the monitoring results of the shipment and inventory of the patient medications.

13. The computer program product of claim 8, wherein a determination is made as to whether patients have been enrolled in the one or more medical trials intended for the patients.

14. The computer program product of claim 8, wherein a determination is made as to whether the monitoring results of the shipment and inventory of the patient medications are more accurate than the patient enrollment in the one or more medical trials.

15. A system comprising:
a memory configured to store instructions;
one or more processors configured to execute the instructions to perform operations to:
receive data images of patient medications from one or more databases;
create a training set using the received data images of patient medications;
train a machine learning system using the training set, wherein the machine learning system is trained to monitor shipment and inventory of the patient medications, patient enrollment in one or more medical trials, and a distribution of the patient medications in the one or more medical trials; and
apply the trained machine learning system with monitoring results of the shipment and inventory of the patient medications, patient enrollment in the one or more medical trials, and the distribution of the patient medications in the one or more medical trials.

16. The system of claim 15, wherein a determination is made as to whether the machine learning system needs to be re-trained based on the monitoring results of the distribution of the patient medications in the one or more medical trials.

17. The system of claim 15, wherein source data verification for clinical research associates is removed.

18. The system of claim 15, wherein the one or more processors determine that the monitoring results of the shipment and inventory of patient medications exceed an accuracy threshold.

19. The system of claim 15, wherein the machine-learning system is re-trained in a plurality of time intervals.

20. The system of claim 15, wherein the monitoring results of the distribution of the patient medications are compared to an accuracy threshold.
